(19) United States
(12) Patent Application Publication
(10) Pub. No.: US 2026/0345472 A1
(43) Pub. Date: Nov. 5, 2026

(54) USE OF AN EXTRACT OF SICHUAN PEPPER FOR A SKIN TREATMENT AND A COMPOSITION SUITABLE FOR THIS USE

(71) Applicant: CRODAROM, Chanac (FR)

(72) Inventor: Amandine Fischer Desnos, Chanac (FR)

(73) Assignee: CRODAROM, Chanac (FR)

(21) Appl. No.: 19/107,999

(22) PCT Filed: Aug. 24, 2023
(86) PCT No.: PCT/IB2023/058397

§ 371 Date: Feb. 28, 2025

(30) Foreign Application Priority Data
Sep. 9, 2022 (FR) FR2209032
Mar. 23, 2023 (FR) FR2302746

(51) Int. Cl. A61K 8/9789; A61K 8/02; A61K 8/04; A61Q 15/00; A61Q 19/00; A61Q 19/08

(52) U.S. Cl. CPC A61K 8/9789; A61K 8/0216; A61K 8/042; A61Q 15/00; A61Q 19/007; A61Q 19/08; A61K 2236/31; A61K 2800/874

(57) ABSTRACT

The present invention concerns the use of a Sichuan pepper extract and a composition comprising said extract, for a cosmetic and/or dermatological treatment, in particular a topical treatment. The extract according to the invention has moisturising, soothing, anti-ageing, anti-odour, and skin care effects on oily skin, including the scalp, and on acne-prone skin, in particular by acting on the production of various molecules such as hyaluronic acid, elastin, collagen and laminins, and on the inhibition of oxidation, radicals, lipases and inflammation factors, while preserving the skin microbiota.

### CROSS REFERENCE TO RELATED APPLICATIONS

This application is the U.S. National Phase of PCT/IB2023/058397, filed Aug. 24, 2023, which claims priority to French Application No. FR 2209032, filed Sep. 9, 2022, and French Application No. FR 2302746, filed Mar. 23, 2023, the disclosures of each of these applications being incorporated herein by reference in their entireties for all purposes.

### FIELD OF THE INVENTION

The present invention relates to the use of an extract of Sichuan pepper, more particularly of berries, and a composition comprising said extract for a cosmetic or dermatological treatment, in particular a topical treatment.

«Cosmetic or cosmeceutical treatment» means a treatment which treats healthy skin and/or skin appendages, said treatment being intended to improve or beautify their appearance and condition. Such treatment has no therapeutic purpose.

### BACKGROUND OF THE INVENTION

The present invention relates in particular to the cosmetic and dermo-pharmaceutical industries, which manufacture and/or use products intended for the treatment of the skin, including the scalp, mucous membranes and appendages (such as hair, eyelashes, eyebrows, nails) of mammals, animals or humans, for improving their appearance and/or their general condition.

These industries are in increasing demand for new products, in particular in demand for new active ingredients which are derived from plants because they offer a combination of efficacy, limitation of risks of irritation and allergy, reduction of side effects and biodegradability with the possibilities of labelling/certification and matching with a logic of sustainable development and/or fair trade.

«Holistic» products appear on the market to enhance the daily skincare routine. The aim is to act on the user's well-being, to deal with its emotions as a whole. The product must give the user a good overall feeling, both in terms of skin beautifying and in terms of sensory and olfactory comfort. Beautifying the skin will typically mean, for example, a smoother skin with less roughness, greater elasticity, less sagging, a more radiant complexion, more homogeneous with fewer imperfections, while sensorial comfort will mean, for example, a smooth touch, a soothing of the skin with less redness and tightness, and olfactory comfort by neutralising or masking body odours.

Human odour is genetically controlled and systematically influenced by gender and ethnicity, along with emotional, physiological and environmental factors (influence of sweat composition and quantity). Body malodour affects social interaction by reducing self-confidence.

Body odour originates from sweat secreted by the sudoriferous glands, which can be of two types:
Eccrine sweat glands are found mainly on the hands palms and the feet soles. Eccrine sweat is responsible for regulating body temperature.
Apocrine sweat glands are bigger, confined to shaggy areas, mainly the armpits, perineal area and scalp. Apocrine sweat is responsible for body odour.

The apocrine sweat contains large, odourless, and non-volatile molecules. Odour is generated when these molecules are lysed into small, volatile odorous entities. This lysis is carried out by enzymes including lipases, which are themselves produced by skin bacteria.

To combat odours, most people use antiperspirant or deodorant on a daily basis. Most of these products are antibacterial and/or contain aluminium salts. These products have the ability to tighten the pores of the skin, and therefore reduce the flow of sweat, but have numerous disadvantages, including an increased risk of breast cancer.

### SUMMARY OF THE INVENTION

The aim of the present invention is to respond to all of these demands, namely to offer a complete cosmetic product and treatment, in particular a “holistic” treatment, and more specifically to respond to the demand for a body odour treatment.

### BRIEF DESCRIPTION OF THE DRAWINGS

FIG. 1 represents a photograph to illustrate the anti-lipase test, showing an agar medium containing glyceryl tributyrate which has not been brought into contact with the extract according to the invention (control case).

FIG. 2 represents a similar photograph, but with contact with the extract according to the invention.

### DETAILED DESCRIPTION OF THE INVENTION

«Body odour treatment» means an anti-odour treatment designed to reduce, mask, eliminate, absorb and/or limit the generation of the bad odour resulting from the decomposition of sweat by bacteria.

For this purpose, the applicant provides the use of a Sichuan berries extract of species Zanthoxylum bungeanum or Zanthoxylum simulans for a non-therapeutic cosmetic treatment of the skin and its appendages. Preferably according to the invention, the treatment is a topical treatment.

«Topical treatment» or «topical use» means according to the invention, an application that is intended to act where it is applied: skin, mucosa and/or appendages.

The Sichuan pepper tree is defined by one of these two species: Zanthoxylum bungeanum and Zanthoxylum simulans. It is from the Sichuan region (China).

The Sichuan pepper tree used according to the invention is preferably Zanthoxylum bungeanum.

It is a thorny and robust shrub of the Rutaceae family, which corresponds to the citrus family. The main ethnobotanical properties attributed to this plant species are the relief of dental problems and gastrointestinal disorders, the effectiveness against rheumatism and its analgesic action. The fruit envelopes of the Sichuan pepper plant are widely used in Japan as a spice for their powerful, aromatic smells and ripe citrus flavors.

Sichuan berries come from the pericarp of the fruit of the Sichuan pepper tree.

In vitro tests results are given below in the description showing beneficial cosmetic activities for the skin and its appendages:
On the olfactory level, a treatment to reduce body odour by reducing the lipase activity of bacteria, while advantageously preserving the skin microbiota, whereas the majority of products currently on the market are antibacterial. An in vivo olfactory sweat test confirms these results.
On the skin beautifying or general improvement:
1) an anti-ageing treatment, for a denser, plumper, firmer, softer and more elastic skin with, as a result, a more even and/or smoother skin texture and a more radiant complexion, thanks to the preservation and stimulation of the synthesis of various molecules of the dermal extracellular matrix (ECM), such as:
elastin, responsible for skin resilience and elasticity, and
collagen VII and laminins strengthening the dermal-epidermal junction (DEJ), which ensures that keratinocytes are well anchored to the basal layer and that there is a better communication between the cells, leading to a polarisation of the keratinocytes and therefore to a better skin barrier. The barrier is therefore less fragile, which contributes to the flexibility of the epidermis.
an increase of the anti-radical and antioxidant effect, particularly on the DPPH (2,2-diphenyl-1-picrylhydrazyl) radical and singlet oxygen, and also an anti-glycation effect, helping to preserve the dermal ECM.

The increase in oxidative and radical forms is linked to age and/or repeated environmental stresses, such as UV rays. Radicals and reactive oxygen species attack membrane lipids and/or functional proteins in the dermal ECM, such as collagen and elastin.

Regarding glycation, it also affects proteins and/or reducing sugars. These interactions with proteins and/or reducing sugars alter the mechanical and elastic properties of the dermal ECM, which becomes less flexible, more rigid, but also more flabby and less reactive. Visually, this results in the appearance of fine lines and wrinkles, and a skin that is dull, flaccid, lacking radiance, tired-looking, and lacking tone and suppleness.
2) a treatment of oily and acne-prone skin, by reducing sebum production and/or an astringent action, which together have the effect of reducing the size of pores and making them less visible, reducing the shiny appearance characteristic of oily skin and/or making the scalp healthier, with less dandruff and associated itching.
3) An anti-dandruff scalp treatment, by reducing sebum production and/or the lipase activity of bacteria, while preserving the skin microbiota.

Sebum leads to excessive growth of dandruff-causing bacteria such as Malassezia fungi on the scalp. This fungus is responsible for the formation of dandruff and the itching associated with it.

On the surface of the scalp, Malassezia release lipases to ensure their growth, which hydrolyse triacylglycerols into monoacylglycerols and then into fatty acids and glycerol. These free fatty acids inhibit the growth of other micro-organisms, encourage the proliferation of Malassezia and penetrate the stratum corneum, causing damage to the skin barrier.
4) a moisturising treatment, with an increase in hyaluronic acid synthesis and in keratinocyte differentiation.

Hyaluronic acid is a major constituent of the epidermis contributing to the barrier function as well as to the maintaining of a satisfactory hydration of the skin. It is capable of absorbing 1,000 times its own weight in water. It is in the form of an aqueous and nourishing gel which fills the spaces between the keratinocytes. It prevents from dryness of the skin, which is known to alter the texture of the skin, giving it a rough touch.

The differentiation of the keratinocytes begins from the basal layer of the epidermis. The epidermis is divided into a plurality of layers:
the deeper layer is the basal layer, comprising keratinocytes, and
the most superficial layer is the stratum corneum, consisting of a plurality of keratinocyte layers at the terminal stage of their differentiation and called corneocytes. This highly water-insoluble corneal envelope is composed of anucleate corneal cells, without cytoplasmic organelles and completely keratinised.

Keratinocytes migrate from the deeper layer to the most superficial layer, differentiating themselves into corneocytes.

The stratum corneum is a semi-permeable protective layer that prevents water loss and keeps the skin hydrated. It is composed of the corneocytes, lipids and lamellar bodies, and “natural moisturizing factors” (NMF-substances capable of binding water in the stratum corneum) which maintain the level of skin moisture at an optimum level.

The transepidermal water loss (TEWL) characterises the evaporation of water contained in the skin: the more the skin barrier is damaged, the more easily water evaporates.

Improved differentiation of keratinocytes leads to a strengthening of the skin barrier and, as a result, leads to a better protection of the epidermis and improved moisturizing.

### on the Sensory Level:

A skin soothing treatment, thanks to the reduction of inflammation mediators responsible for the appearance of skin micro-inflammations causing feelings of tightness and discomfort, and also thanks to a moisturizing treatment as described above, resulting in a skin that is softer and less rough.

### On an Emotional Level:

A cosmetic treatment adapted to improve consumer well-being by increasing feelings of happiness and enthusiasm, and reducing stress and annoyance, as demonstrated by an in vivo test using electroencephalogram (EEG) analysis.

These cosmetic effects can be envisaged according to the invention separately or in combination, making it possible in particular to offer a use of “holistic” type combining olfactory, beautifying, sensorial and emotional effects.

Thus, preferably the present invention provides the use of Sichuan berry extract of the species Zanthoxylum bungeanum or Zanthoxylum simulans, for at least one treatment selected from:
a body odour treatment; and/or
a treatment of oily and acne prone skins; and/or
an anti-ageing treatment; and/or
a moisturizing treatment; and/or
a soothing treatment.

According to other preferred features, the extract of Sichuan berries, of the species Zanthoxylum bungeanum or Zanthoxylum simulans, used according to the invention can be obtained by the usual solid/liquid extraction techniques including, for example, maceration, simple decoction, infusion, leaching, extraction under reflux, extraction by subcritical or supercritical fluid, extraction by means of ultrasound or microwaves or any other physical and/or chemical method, such as percolation, digestion, cryoextraction, enzymatic digestion, etc.

Preferably according to the invention, the extract is obtained by extraction under reflux, maceration, decoction, infusion, leaching, extraction by subcritical or supercritical fluid, extraction by means of ultrasound or microwaves, percolation, digestion, cryoextraction, or enzymatic digestion.

«Maceration» means a process consisting of soaking a plant or part of a plant in an extraction solvent at room temperature.

«Infusion» means a process consisting of bringing the extraction solvent to the boil before pouring it onto the plant or part of the plant.

«Decoction» means a process consisting of blending the plant or the part of plant with the extraction solvent, then heating the mixture at the boiling point.

«Digestion» means a process consisting of blending the plant or the part of plant with the extraction solvent, then heating the mixture below boiling point.

Preferably, according to the invention, the extract is obtained by digestion of Sichuan berries in at least one aqueous, alcoholic, hydroalcoholic, glycolic or lipidic solvent or mixture thereof, such as water, a C1, C2, C3 or C4 alcohol, or a polyol selected from pentanediol, sorbitol, butylene glycol, pentylene glycol, propylene glycol, hexanol, caprylic/capric triglyceride (GTCC), a vegetable oil, or a mixture thereof.

Preferably, the extraction solvent is a glycolic solvent, and more preferably, the propylene glycol.

The extract of Sichuan berry, Zanthoxylum bungeanum or Zanthoxylum simulans, used according to the invention comprises as main components xanthoxylin, polyphenols including flavonoids, carbohydrates, and proteins.

This extract can be used as it is or in a composition, diluted in a physiologically acceptable medium. The nature of the medium is defined as a function of the properties of the Sichuan berry extract, and also as a function of the destination of the composition formed: simple ingredient, more sophisticated galenic form of a final composition for the consumer.

“Physiologically acceptable medium” means according to the present invention, without limitation, an aqueous or hydro-alcoholic solution, a water-in-oil emulsion, an oil-in-water emulsion, a micro-emulsion, an aqueous gel, an anhydrous gel, a serum, a dispersion of vesicles, or a powder.

“Physiologically acceptable” means that the compositions are suitable for topical or transdermal use, in contact with mucous membranes, appendages (nails, hair and body hair), scalp and skin of mammals, particularly human, compositions which may be ingested, or injected into the skin, without risk of toxicity, incompatibility, instability, allergic response, and others. This “physiologically acceptable medium” forms what is commonly called the excipient of the composition.

According to the invention, the physiologically acceptable medium can be an aqueous, hydroglycolic or hydroalcoholic solution, or a water-in-oil emulsion, an oil-in-water emulsion, or a microemulsion. Preferably, it is hydroglycolic. Preferably, the physiologically acceptable medium is a blend of propylene glycol and water.

Therefore, the present invention covers a non-therapeutic, cosmetic or nutraceutical topical treatment method for beautifying or improving the appearance and general condition of skin and/or its appendages, and for treating their imperfections, by applying in a subject in need thereof an effective amount of at least one Sichuan berry extract according to the invention or a composition comprising it, in a physiologically acceptable medium.

The effective amount of Sichuan berry extract according to the invention, that is to say its dosage, depends on the destination of the composition. It depends on various factors, such as the age, the condition of the patient, the severity of the disorder and the administration mode. An effective amount means a non-toxic amount enough to achieve the desired effect.

In a cosmetic composition according to the invention containing at least a Sichuan berry extract, to be present in an effective amount, it is generally present in an amount ranging from 0.000001% and 15% based on the total weight of the composition, preferably ranging from 0.00001% and 10%, depending on the destination of the composition and the more or less pronounced desired effect. More preferably, the effective amount is between 0.0001% and 5% based on the total weight of the composition.

All percentages and ratios used herein are by weight of the total composition and all measurements are made at 25 °C unless it is otherwise specified.

According to other features, the cosmetic treatment method according to the invention can be combined with one or more other treatment methods targeting the skin such as luminotherapy, heat or aromatherapy treatments.

Devices with several compartments or kits may be proposed to apply the method described above which may include for example and non-restrictively, a first compartment containing a composition comprising the Sichuan berry extract of the invention, and in a second compartment an additional active ingredient, the compositions contained in the said first and second compartments in this case being considered to be a combination composition for simultaneous, separate or stepwise use in time, particularly in one of the treatment methods recited above.

For indication, for a cosmetic face treatment, the European standard dosage of a cream is 2.72 mg/cm²/day/person and for a cosmetic body treatment the European standard dosage of a lotion is 0.5 mg/cm²/day/person.

According to other advantageous features, the Sichuan berry extract according to the invention may be associated with one or more other active ingredients at effective concentrations that can act synergistically or additionally for reinforcing and achieving the desired effects described for the invention, such as the following agents: filtering radiations, in particular UVA, UVB, IR or generated by blue light, hydrating, moisturizing, humectant, calming, muscle relaxant, slimming, restructuring, firming, replumping, lifting, smoothing, acting on blood microcirculation, inflammation, free radicals, anti-aging, anti-fine lines and wrinkles, lightening, acting on complexion, anti-glycation, anti-carbonylation, pro-pigmenting, acting on stratum corneum, on dermal-epidermal junction, on HSP protein production, on firmness, elasticity and tone of skin, on hair growth or anti-regrowth (including eyelashes and eyebrows), on eye contours (dark circles and under eye bags), peptides, vitamins, etc. These active ingredients can be obtained from plant materials, such as plant extracts or products of plant culture or fermentation production methods.

The Personal Care Products Council (“International cosmetic ingredient dictionary & handbook” published by the “Cosmetic, Toiletry, and Fragrance Association, Inc.”, Washington, D.C.) describes a non-limited wide variety of cosmetic and pharmaceutical ingredients conventionally used in the skin care industry that can be used as additional ingredients in the compositions for the present invention, as long as they are physically and chemically compatible with the other ingredients of the composition and especially with the active ingredients of the present invention. Also, the nature of these additional ingredients should not unacceptably alter the benefits of the active ingredient of the invention. These additional ingredients can be synthetic or natural such as plants extracts or issued from a bio-fermentation process.

Further skin care actives that are particularly useful combined with the composition can be found in Sederma's commercial literature and on the website www.sederma.fr, in Croda's commercial literature and on the website www.croda.fr.

More specifically, the Sichuan berry extract according to the invention can be combined with at least one compound chosen among vitamin compounds, group B C, E, F, D and A, especially compounds as niacinamide or tocopherol, retinoids compounds as retinol, hyaluronic acid, hexamidine, α-lipoic acid, resveratrol or DHEA, peptides, aluminium salts, phosphate salts, magnesium oxide, zirconium hydrochloride salts, ceramides, which are classic active ingredients used in topical cosmetic or dermo-pharmaceutical compositions.

Furthermore, the present invention provides the use of a Sichuan berries extract of species Zanthoxylum bungeanum or Zanthoxylum simulans, as described above, for the manufacture of a composition for a cosmetic treatment, as described above.

The composition for the use according to the invention can be provided in any galenic form (examples are given below) defined according to the composition destination and application site.

A composition according to the invention can be applied to the face, body, neckline, scalp, hair, eyelashes, body hair, in any form or vehicle known to those skilled in the art, in particular in the form of a solution, dispersion, emulsion, paste or powder, individually or as a premix or vehicle individually or as a premix in a bound form, incorporated or adsorbed in vectors such as macro-, micro-, or nanocapsules, macro-, micro- or, nanospheres, liposomes, oleosomes or chylomicrons, macro-, micro-, or nanoparticles or macro-, micro or nanosponges, micro- or nanoemulsions, or adsorbed on organic polymer powders, talcs, bentonites, spores or exines, and other inorganic or organic supports.

In cosmetics, applications can be proposed in particular in the ranges of skin care for the face, body, hair and body hair and ranges of make-up treatments, in particular eyelashes and eyebrows.

For example, the galenic form of the composition can be a lotion, a cream, a butter, a milk, a solid form, a foam, a gel, a deodorant, an antiperspirant, a shampoo, a conditioner, a hair mask, a face mask, a shower gel, etc.

Also for example, in the case of an anti-odour use, the galenic form of the composition can be a cream for the face, the feet, and/or the whole body, a spray, an aerosol, a roll-on or a deodorant stick. For an «holistic» product, the preferred galenic form may be a body lotion.

The galenical formulations can enter in different product ranges for personal care and/or beauty products including skin care, cleaning, makeup, cleansing, sunscreen, artificial tanning, pre-shave, shaving or aftershave, moisturizer, humectant, emollient, conditioning, exfoliating, astringent, depilatories or antiperspirant, deodorant, etc.

The composition may be incorporated onto a non-woven or woven material, with natural or synthetic fibres, wool, or any material intended to come into contact with skin and that can be used in clothing, including tights and socks, shorty, day or night underwear, tissues, handkerchiefs or fabric to exert its cosmetic effect via the contact skin/textile and enable continuous topical delivery (cosmetic-textiles).

According to the invention, it is thus also provided a woven or non-woven fabric comprising at least one a Sichuan berry extract, for use in a non-therapeutical cosmetic treatment.

The present invention will be better understood in the light of the following description of an embodiment, the in vitro tests and figures described below.

### Examples of Obtaining a Sichuan Berry Extract According to the Invention and a Composition Comprising it

Plant material: Sichuan berries of the species Zanthoxylum bungeanum.

Maceration solvent: Propylene glycol

Protocol: berries are macerated in propylene glycol for 1 to 6 hours at 70 to 100 °C, then the macerate is filtered to remove solid plant debris. Several successive filtrations are carried out, with a size of between 5 and 0.1 μm. The filtered macerate constitutes the berry extract according to the invention. Advantageously, according to this method a xanthoxylin-rich extract of Sichuan berries is obtained, with a xanthoxylin content of between 10 and 20% by weight based on the weight of the dry extract. This molecule is identified by high-performance thin-layer chromatography (HPTLC).

To obtain a composition comprising the extract according to the invention, the filtered macerate obtained is mixed with a physiologically acceptable matrix consisting of a water-propylene glycol mixture. The ratio of water to propylene glycol in the composition is between 0:100 and 30:70.

### Evaluation of the Various Activities of the Extract According to the Invention, by In Vitro and In Vivo Tests

a. Body Odours
i. Lipase Activity

### Principle

Lipases are able to hydrolyse triglycerides having long fatty acid chains into glycerol and the corresponding volatile fatty acids responsible for unpleasant odour.

Lipases are widely distributed in bacteria, yeasts and filamentous fungi. They are produced by Gram-positive bacteria such as Corynebacterium, Propionibacterium, Cutibacterium and Staphylococcus, as well as Gram-negative bacteria such as Pseudomonas.

In the axillae area, the genus Corynebacterium is mainly responsible for axillary malodour. Gram-negative bacteria bring almost no contribution to axillary malodour.

### Protocol

Glycerol tributyrate substrate (fat-soluble) is hydrolysed by lipases into glycerol and butyric acid (water-soluble). When the fat-soluble substrate is mixed with the liquid medium or deposited on the agar, the culture medium becomes cloudy. When the substrate becomes water-soluble following the action of the lipase, this cloudiness of the culture medium disappears in proportion to the hydrolysis.

Two methods are used to demonstrate the anti-lipase activity of the extract according to the invention:
a method in liquid medium for which the cloudiness is measured at 620 nm by spectrophotometry.
a method on an agar plate, for which the cloudiness is assessed visually.

### Results

### 1. Analysis in Liquid Medium

Evolution of the lipase activity of Corynebacterium xerosis over time. Effect of a composition comprising 3% of the extract according to the invention compared to the control (n=3):

TABLE 1

| | | | 3% of the extract | |
|---|---|---|---|---|
| | | | according to the | Variation (%); |
| | | Control | invention | significance |
| 0 | hour | 0.00 ± 0.00 | 0.00 ± 0.00 | 0.0%; nsd* |
| 48 | hours | 57.00 ± 0.06 | 43.70 ± 1.31 | −23.3%; p < 0.0001 |
| 72 | hours | 77.40 ± 0.19 | 40.50 ± 0.20 | −47.7%; p < 0.0001 |

*nsd: non-significant data

These results show that 3% of the extract according to the invention significantly reduces the lipase activity of Corynebacterium xerosis at 48 hours (−23.3%; p<0.0001) and 72 hours (−47.7%; p<0.0001).

### 1. Analysis in Agar Medium

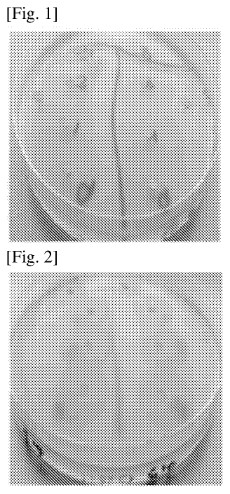

FIG. 1 represents a photograph of the agar medium containing glycerol tributyrate that has not been in contact with the extract according to the invention (control case), after 48 hours at 37 °C under anaerobic conditions.

FIG. 2 represents a photograph of the agar medium containing glycerol tributyrate that has been in contact with 3% of the extract according to the invention, after 48 hours at 37 °C under anaerobic conditions.

By comparing these two figures, we can see that the cloudiness is less present in the agar medium containing 3% of the extract according to the invention than in the control case, which means that the glyceryl tributyrate has been hydrolysed to a greater extent and that the extract according to the invention therefore reduces the lipase activity of Corynebacterium xerosis under the test conditions.

### Conclusion

Both methods, in liquid and in agar medium, demonstrate that the lipase activity of Corynebacterium xerosis decreases in contact with the extract according to the invention.

i. Protection of Bacterial Flora

### Principle

Corynebacterium xerosis and Staphylococcus epidermis are important commensal strains of the axillary area. Staphylococcus epidermis is a beneficial probiotic skin strain and Corynebacterium xerosis is the main strain responsible for malodour.

The Minimal Inhibitory Concentration (MIC) is the lowest concentration of an antibacterial agent needed to inhibit the visible growth of the strain being tested. The higher the percentage is, the less the tested product has antibacterial activity.

### Protocol

The culture medium is inoculated with the strains Corynebacterium xerosis and Staphylococcus epidermis, and different percentages of the extract according to the invention or inoculated with a product on the market known for its antibacterial action (Triclosan or Phenonip™). Then, the test is incubated for 48 hours at 32.5 °C.±2.5 °C. After incubation, the presence or absence of cloudiness is observed, with the presence of cloudiness indicating a bacterial growth.

### Results

Minimal Inhibitory Concentration (MIC) of the extract according to the invention, compared to two products on the market (n=3).

TABLE 2

| | MIC |
|---|---|
| Extract according to the invention | |
| Corynebacterium xerosis | 14% |
| S. epidermidis | >20% |
| Triclosan | |
| Corynebacterium xerosis | >0.05% |
| S. epidermidis | 0.013% |
| Phenonip ™ (Phenoxyethanol (and) | |
| Methylparaben (and) Ethylparaben | |
| (and) Propylparaben (and) | |
| Butylparaben (and) Isobutylparaben) | |
| Corynebacterium xerosis | 0.63% |
| S. epidermidis | 0.63% |

These results show that Triclosan and Phenonip™, both preservatives used in cosmetic products, have a very low MIC, respectively 0.013% and 0.63% on S. epidermidis, whereas the extract according to the invention has a MIC greater than 20%.

In the same way, Phenonip™ shows a MIC of 0.63% on Corynebacterium xerosis whereas the extract according to the invention has a MIC of 14%.

### Conclusion

Therefore, the extract according to the invention is a product that respects the microbiome, because a high concentration is needed to obtain an equivalent result of 0.63% of Phenonip™ or 0.013% of Triclosan and alter the two most important microbial strains of the axillary area. Therefore, the extract according to the invention does not alter bacterial growth.

i. In Vivo Olfactory Evaluation of Sweat

### Protocol

The study was carried out on 14 volunteers: 3 men and 11 women. Among the panellists 5 experts took part in the study. They are trained and experienced to odour and perfume evaluation.

The intensity of axillary odour is assessed every hour, from 0 to 8 hours after application of a composition comprising 3% of the extract according to the invention or a placebo. The study took place over 3 days: the first day for odour cleansing, the second day with the placebo application on one side and the composition comprising 3% of the extract according to the invention on the other side, and the reverse on the third day.

Volunteers were asked, one day before the trial and until the end of the third day, not to eat spicy foods, onions, or garlic, and not to use scented products.

Once the product had been applied, the smell was assessed every hour and a score was given for each armpit, ranging from 0 to 5:0 (no pleasant smell), 1 (very slight pleasant smell), 2 (slight pleasant smell), 3 (normal smell), 4 (pleasant smell) and 5 (very pleasant smell).

### Results

Variation of odour intensity depending on time. Effect of a composition containing 3% of the extract according to the invention compared to the same composition without the extract according to the invention (placebo):

TABLE 3

| | Panel of volunteers (mean) | | Expert panel (mean) | |
|---|---|---|---|---|
| | T = 0 hour | T = 8 hours | T = 0 hour | T = 8 hours |
| Placebo | 4.7 ± 0.55 | 1.7 ± 0.71 | 4.5 ± 0.53 | 2.1 ± 0.53 |
| 3% of the extract according | 4.9 ± 0.31 | 2.3 ± 0.94 | 5.0 ± 0.00 | 3.3 ± 0.00 |
| to the invention | | | | |
| Variation (%); significance | −4.6%; p < 0.01 | −35.4%; p < 0.01 | −11.1%; p < 0.01 | −57.1%; p < 0.01 |

These results show that for the panel of volunteers, the composition comprising 3% of the extract according to the invention significantly reduces the bad axillary odour after 8 hours of application (−35.4%; p<0.01).

These results show that for the expert panel, the composition comprising 3% of the extract according to the invention significantly reduces the bad axillary odour after 8 hours of application (−35.4%; p<0.01).

### Conclusion

These results show that the extract according to the invention decreases body odour in the axilla area.

a. Anti-Ageing
i. Elastin Synthesis

### Principle

Elastin is an extracellular matrix molecule. The elasticity of the skin is modified during aging due to the decrease in the quantity of produced elastin, due to an inappropriate assembly of the elastin fibres.

### Protocol

The cells as they reached confluency, are brought into contact or not (for the control cases) with the extract according to the invention in culture medium, then incubated at 37 °C and 5% CO₂ for 5 days. After this contact, cell layers are rinsed, fixed, and stained with anti-elastin antibody. The labelling is revealed using a fluorescent secondary antibody and photographs captured under a microscope. Elastin production can be quantified by image analysis on these photographs. A counter-labelling of the nuclei is performed using the fluorescent dye HOESCHT, which marks the DNA, to evaluate the cell population and thus to weight the fluorescence data obtained.

### Results

Variation of elastin production by fibroblasts. Effect of the extract according to the invention at 0.14% compared to the control (n=3):

TABLE 4

| | Elastin (Mean | |
|---|---|---|
| | area/number of | Variation (%); |
| | cells) | significance |
| Control | 103 ± 17 | Reference |
| 0.14% of the extract according | 249 ± 13 | +141%; p < 0.01 |
| to the invention | | |

These results show that 0.14% of the extract according to the invention significantly increases by 141% (p<0.01) the production of elastin by fibroblasts.

i. Strengthening of the Dermo-Epidermal Junction

### Principle

DEJ assures the cohesion between the dermis and the epidermis. During aging, a decrease of synthesis of its components (especially collagen VII and laminin) is observed. The aging of the DEJ has significant repercussions on the resilience of the skin and the loss of its dynamism.

### Protocol

Human keratinocytes (HK) were cultured at sub-confluence and were brought into contact or not (for the control cases) with the extract according to the invention. After this contact, the syntheses of collagen VII and laminins are evaluated using ELISA type kits. The number of cells is estimated using the Hoechst method and used to standardize the results.

### Results

Variation of collagen VII production by keratinocytes. Effect of the extract according to the invention at 0.26% compared to the control (n=5):

TABLE 5

| | Collagen VII | Variation (%); |
|---|---|---|
| | (μg/mL/10⁶ cell.) | significance |
| Control | 5.8 ± 0.3 | Reference |
| 0.26% of the extract | 8.5 ± 0.4 | +47%; p < 0.01 |
| according to the invention | | |

These results show that 0.26% of the extract according to the invention significantly increases by 47% (p<0.01) the production of collagen VII by keratinocytes.

Variation of laminins production by keratinocytes. Effect of the extract according to the invention at 0.26% compared to the control (n=5):

TABLE 6

| | Laminin | Variation (%); |
|---|---|---|
| | (μg/mL/10⁶ cell.) | significance |
| Control | 32 ± 2 | Reference |
| 0.26% of the extract | 51 ± 3 | +58%; p < 0.01 |
| according to the invention | | |

These results show that 0.26% of the extract according to the invention significantly increases by 58% (p<0.01) the production of laminins by keratinocytes.

### Conclusion

These results show that the extract according to the invention has a direct action of strengthening the DEJ by stimulating the synthesis of laminins and collagens VII.

The extract according to the invention can improve the aging of the skin linked to a disorganization of the DEJ, by counteracting the loss of suppleness and elasticity that it causes.

i. Antioxidant Capacity

### Principle

Oxidative stress plays a central role in the cutaneous response to various stresses. Free radicals (H₂O₂, OH·, O₂—, O₂, ¹O₂ . . . ) lead to protein, lipid, and DNA damages, causing premature aging of the skin.

### Protocol

For the DPPH test: DPPH (2,2-diphenyl-1-picrylhydrazyl) is a stable free radical which is widely used for the detection of free radical scavengers. This molecule while losing its radical character is converted into DPPH-H (2,2-dipheny-1-picrylhydrazine). This conversion is accompanied by discoloration by oxidation-reduction, from violet to yellow, which can be followed in time by spectrophotometry at 490 nm.

For the O₂ singlet test: A system generating O₂ singlet receives the product according to the invention. The degradation of uric acid is followed at 292 nm by spectrophotometry.

### Results

Variation of DDPH production (n=6). Effect of 2 or 3% of the extract according to the invention compared to the control:

TABLE 7

| | DDPH | |
|---|---|---|
| | (Average variation of | Variation (%); |
| | optical density) | significance |
| Control | 0.268 ± 0.005 | Reference |
| 2% of the extract | 0.101 ± 0.037 | −62%; p < 0.01 |
| according to the invention | | |
| 3% of the extract | 0.082 ± 0.059 | −69%; p < 0.01 |
| according to the invention | | |

These results show that the extract according to the invention significantly decreases the presence of DPPH.

Variation in singlet O₂ production (n=2). Effect of 2 or 3% of the extract according to the invention compared to the control:

TABLE 8

| | Singlet O₂ | |
|---|---|---|
| | (Average variation of | Variation (%); |
| | optical density) | significance |
| Control | 32.33 ± 0.81 | Reference |
| 2% of the extract according | 20.02 ± 0.95 | −38%; p < 0.01 |
| to the invention | | |
| 3% of the extract according | 14.87 ± 1.49 | −54%; p < 0.01 |
| to the invention | | |

These results show that the extract according to the invention significantly decreases the presence of singlet O₂.

### Conclusion

The extract according to the invention has a high antioxidant capacity, allowing to effectively combat premature skin ageing.

i. Non-Enzymatic Glycation of BSA

### Principle

Glycation of proteins with reducing sugars in the skin is also responsible for skin aging.

The formation of many glycated proteins whose functional, enzymatic, structural properties are altered, has consequences on the correct functioning of the cell or the organism.

The consequence is the altering of the mechanical and elastic properties of the extracellular matrix of the dermis, which becomes less flexible, more rigid, but also more flaccid and less reactive, also resulting in a dull complexion.

### Protocol

The study of the non-enzymatic glycation is done between a model protein, the serum albumin, which serves as a target, and an edible reducing sugar from fruits. The protein is gradually glycated (bound to sugar) in an irreversible manner, in the presence or not of the product according to the invention. This change is followed by fluorescence.

### Results

Variation of glycation (n=2). Effect of 2 or 3% of the extract according to the compared to the control:

TABLE 9

| | Non-enzymatic glycation | |
|---|---|---|
| | (Average variation in | Variation (%); |
| | fluorescence unit) | significance |
| Control | 84540 ± 1803 | Reference |
| 2% of the extract according | 7926 ± 403 | −92%; p < 0.01 |
| to the invention | | |
| 3% of the extract according | 5304 ± 667 | −95%; p < 0.01 |
| to the invention | | |

The results show the strong anti-glycant potential of the extract according to the invention.

### Conclusion

The extract according to the invention has anti-glycation capacity that also helps to combat skin ageing.

a. Oily and/or Acne Prone Skins
i. Astringency

### Principle

An astringent substance, able to shrink or tighten body tissues by aggregation, is perfectly adapted to:
Tighten enlarged pores and reduce excessive oil and sweat secretion;
Remove sebum excess;
Precipitate amino acids causing body malodour. These amino acids are formed from corneocytes, secreted proteins or free amino acids generated by bacterial catabolism

The in tubo assay for determining the astringency capacity of a product is based upon tannin-polymer interaction resulting in the formation of insoluble polymer tannin complexes which then precipitate. The difference in optical density (OD) before and after precipitation corresponds to the astringency capacity of the product.

### Protocol

The extract according to the invention on the one hand, and red wine on the other, are incubated with and without methylcellulose and the optical density at 280 nm is measured by spectrophotometer. The precipitated part of the extract corresponds to the astringent tannin fraction.

### Results

Astringency of the extract according to the invention compared to red wine (n=4 for the extract according of the invention and n=12 for red wine):

TABLE 10

| | Means Tannins precipitated with | |
|---|---|---|
| | Methylcellulose (MCP) (ppm | |
| | epicatechin equivalent) | % Variation |
| Tannic red wine | 1 334 ± 222 | Reference |
| Extract according to the | 5 276 ± 161 | 295%* |
| invention | | |

Tannic red wine is considered as an astringent positive control. Its astringency is of 1334 ppm. The extract according to the invention shows an astringent capacity that is, statistically different 295% higher than that of tannic red wine.

i. Anti-seborrheic

### Principle

The skin has numerous pores on its surface whose function is to evacuate excess sebum and impurities from the skin, such as dead cells and sweat. Oily skin is associated with excessive sebum production by the sebocytes. Too much sebum leads to changes in the properties of the skin and scalp, for example by increasing the formation of pimples and blackheads and clogging pores, which then become dilated and more visible, leading to uneven skin texture and/or excessive growth of the bacteria responsible for dandruff, such as Malassezia fungi on the scalp. An anti-seborrheic cosmetic active ingredient will counteract this development by reducing sebum production, which will have the effect of tightening skin pores, smoothing it and reducing the oily/shiny appearance with irregular texture characteristic of oily skin and/or making the scalp healthier, with less dandruff and associated itching.

### Protocol

Sebocytes are seeded in their growth medium. At confluence, the cells are contacted or not (control case) with the extract according to the invention for 48 hours. After removing media, monolayers are incubated with Nile Red marker of intracellular lipids which estimates the amount of lipids in the cells. The estimate of the viability is performed in parallel on the same layers using a fluorescent dye.

### Results

Variation of lipidic synthesis by sebocytes. Effect of the extract according to the invention at 2% compared to the control (n=2):

TABLE 11

| | | Variation (%); |
|---|---|---|
| | (UFA/10⁶ cell) | significance |
| Control | 160962 ± 6691 | Reference |
| 2% of the extract according to the | 77026 ± 5163 | −52%; p < 0.01 |
| invention | | |

These results show that exposing sebocytes to 2% of the extract according to the invention reduces the quantity of lipids in sebum-producing cells.

### Conclusion

The extract according to the invention can be used to treat skin disorders associated with oily skin or skin with a tendency to become oily, such as a shiny, glossy appearance and the size and number of pores, to give the skin a smoother, more uniform, more harmonious appearance and/or to make the scalp healthier, with less dandruff and associated itching.

a. Skin Hydrating
i. Hyaluronic Acid Synthesis

### Protocol

Human keratinocytes are cultured at sub-confluence and then contacted or not (for the control cases) with the extract according to the invention. After this contact, the synthesis of hyaluronic acid is assayed using ELISA methods. The cell concentration is estimated using the Hoechst method.

### Results

Variation of hyaluronic acid production by keratinocytes. Effect of the extract according to the invention at 0.26% compared to the control (n=5):

TABLE 12

| | Hyaluronic acid | Variation (%); |
|---|---|---|
| | (μg/mL/10⁶ cell.) | significance |
| Control | 340 ± 37 | Reference |
| 0.26% of the extract according to the | 1215 ± 106 | +257; p < 0.01 |
| invention | | |

These results show that the extract according to the invention significantly increases hyaluronic acid synthesis by keratinocytes.

i. Keratinocytes Differentiation

### Protocol

Human keratinocytes are cultured at sub-confluence and then contacted or not (for the control cases) with the extract according to the invention. Differentiation is monitored visually by observing the phenotype of the keratinocytes over 4 days.

### Results

TABLE 13

| | Pro-differentiation effect observed |
|---|---|
| Condition | after 4 days |
| Control | 0 |
| 3% of the | ++ |
| product | |
| according to | |
| the invention | |

Visually, in the control case, the untreated cells show a layer of contiguous keratinocytes with well-marked cell outlines, whereas in the presence of 3% of the product according to the invention, the cells shrink and the contacts between the cells decreases until there are empty spaces between the cells, an aspect characteristic of advanced differentiation of keratinocytes.

### Conclusion

The extract according to the invention increases keratinocyte differentiation, helping to protect the epidermis and improve hydration.

a. Soothing Skin

### Principle

Inflammatory mediators are very present in micro-inflammatory phenomena. Reducing the presence of these mediators has the effect of decreasing sensations of discomfort and redness in sensitive skin.

### Protocol

Normal Human Dermal Fibroblasts (NHDF) are cultured at confluence. At this stage, they are, contacted with the products to test for 24 hours. After this contact period, the cells are irradiated with UVB, and the cells are then contacted with the products to test for another 24-hour period. The quantities of PGE2 and IL-6 synthesized are measured in the culture supernatants by ELISA assay. The number of cells is assessed in order to normalize the data. An analysis of variance and a Student t-test for non-paired series are performed to evaluate the significance of the results.

### Results

Variation of IL-6 and PGE2 production by fibroblasts. Effect of the extract according to the invention at 3% compared to the control (n=3):

TABLE 14

| | | IL-6 (pg/10⁶ | PGE₂ (pg/10⁶ |
|---|---|---|---|
| | | cell) | cell) |
| Not | Control | 1244 ± 32 | 1613 ± 49 |
| irradiated | 3% of the extract according | 627 ± 54 | 334 ± 35 |
| | to the invention | | |
| | Variation (%); significance | −50%; p < 0.01 | −79%; p < 0.01 |
| Irradiated | Control | 9238 ± 323 | 8539 ± 683 |
| with | 3% of the extract according | 3399 ± 181 | 1053 ± 208 |
| UVB | to the invention | | |
| | Variation (%); significance | −63%; p < 0.01 | −88%; p < 0.01 |

Thus, advantageously, the extract according to the invention strongly and significantly reduces both pro-inflammatory messengers.

### Conclusion

These results show that the extract according to the invention reduces skin micro-inflammations. The extract according to the invention can therefore be used to soothe the cutaneous discomforts of sensitive skin such as redness, tightness, etc.

a. In Vivo Assessment of Improved Well-being Using EEG Analysis

### Principle

A lot of research shows that the beauty and quality of the skin are closely linked to emotional well-being, and conversely, emotional well-being is improved when people are satisfied with the beauty and quality of their skin.

Undesirable skin effects, such as sensitive or dry skin, redness, tightness, acne, increased sweating and/or the appearance of unpleasant odours, may be linked to intrinsic, psychological factors such as stress or a hormonal factor, or extrinsic factors such as a reduction in quality of life.

For a “holistic” approach to wellness body care, it is interesting to positively boost the user emotions through the application of a care product.

Emotions result from communication between the neurons in our brain. This communication induces brain waves with a frequency of between 1 Hertz and 80 Hertz, with amplitudes of 10 μV to 100 μV, which can be recorded by an electroencephalograph using electrodes placed at four points in the prefrontal cortex.

The frequency of these waves is known to be linked to the consumer psychological state. Therefore, they can be used to detect emotional states of mind, more specifically by observing alpha waves (8-12 Hz) and beta waves (12-30 Hz). It has been shown that alpha waves predominantly appear when the subject is relaxed or at rest, and can therefore be correlated with brain inactivity, and that beta waves appear when the subject is active, and, therefore, can be correlated with excitement, for example intense mental activity or a stress state.

### Protocol

The study was carried out on 13 healthy volunteers aged over 18 years old: 2 males and 11 females. Unlike the in vivo olfactory test described above, the panellists were not trained or accustomed to carrying out this type of test.

The study was carried out in 5 stages:
1, 2 hours before the start of the session, panellists are asked to wash their forearms with gentle soap.
2. The panellists are asked to relax in order to obtain a quality EEG.
3. The EEG recording is started before the cream is applied. The panellists must apply the cream until it is absorbed by the skin.
4. Then, the panellists are asked to stroke the cream application site.
5. The recording is stopped.

As odours are known to influence emotions by recalling memories, it is requested not to smell the product before, during or after the application in order not to distort the EEG results.

Cream used for the study: cream described in example 4 below vs. placebo cream. The placebo cream used is identical to example 4 below, except that it does not contain the extract according to the invention, although it does contain the solvents used for the extract according to the invention.

The panellists also carried out a sensory evaluation of the cream containing the extract according to the invention vs. placebo. It was found that the creams could not be distinguished sensorially (firmness, stickiness, rigidity, wettability, spreadability, thickness, slipperiness, etc.).

Both analysis (EEG and sensory evaluation) were not carried out on the same day.

The classification of emotions can be based on a dimensional model of valence and arousal. Russell's two-dimensional model was used.

Valence, corresponding to the subject's degree of satisfaction and well-being (‘pleasant’ when positive and ‘unpleasant’ when negative), and arousal (‘excited’ when positive and ‘calm’ when negative) constitute the abscissa and ordinate of the two-dimensional model respectively.

To determine valence, the difference in the ratio of alpha/beta waves between the two cortical hemispheres is calculated. It has been shown that left frontal inactivation is often linked to a negative emotion and right frontal inactivation to a positive emotion.

To determine arousal, the ratio of beta to alpha waves is calculated. As indicated above, beta waves are associated with a state of alertness or excitement, while alpha waves are more dominant in a state of relaxation.

Russell's two-dimensional model is divided into 4 parts:
Q1, when valence is higher than-0.006 and arousal is higher than 0.914. The corresponding emotions are happiness, excitement, euphoria, or joy.
Q2, when valence is higher than-0.006 and arousal is lower than 0.914. The corresponding emotions are satisfaction, concentration, comfort, relaxation, or calmness.
Q3, when valence is lower than-0.006 and arousal is less than 0.914. The corresponding emotions are drowsiness or boredom.
Q4, when valence is lower than-0.006 and arousal is higher than 0.914. The corresponding emotions are stress, annoyance, or nervousness.

### Results

Variation of valence and arousal. Effect of the cream including 2% of the extract according to the invention compared to the placebo cream:

TABLE 15

| | Average | Average | Parts of Russell's |
|---|---|---|---|
| | valence | activation | circular model |
| Placebo cream | −0.0180 | 0.917 | Q4 |
| Cream containing the extract | −0.0049 | 0.915 | Q1 |
| according to the invention | | | |

These results show that the cream including the extract according to the invention increases valence and decreases arousal compared to the placebo cream. This means that valence evolves towards more pleasant emotions and arousal evolves towards calmer emotions.

The cream including the extract according to the invention instantly increases the well-being of volunteers by increasing the feeling of happiness and enthusiasm and reducing feelings of annoyance and stress. It also reduces perspiration and the associated unpleasant odours.

### A. Example of a Galenic Formulation

Various cosmetic formulations are described below, including the extract according to the invention, in particular as formulated in the active ingredient described in point B. above.

Additional active ingredients, acting in support and/or in addition to the activity of the active ingredient according to the invention may be added in the appropriate phase according to their hydrophobic, hydrophilic or amphiphilic nature. These ingredients can be of any category according to their function(s), the place of application (scalp, face, body, neck, bust, hands, etc.), the desired end effect and the targeted consumer, for example specific anti-ageing, anti-wrinkle, moisturizing, anti-dark circles, firming, anti-glycation, slimming, volumizing, soothing, myo-relaxing, anti-redness, sensitive skin, anti-stretch marks, detoxifying, mattifying etc.

Active ingredient according to the invention: Sichuan berry extract according to the invention in a water-propylene glycol matrix.

This ingredient is recommended in a composition at a level of 1 to 5%, preferably 3%. These levels can vary without departing from the scope of the present invention, depending on the more or less pronounced effects sought.

### Example 1: Roll-on Deodorant

TABLE 16

| | | |
|---|---|---|
| Raw materials | % | INCI name |

Phase A

| | | |
|---|---|---|
| SP CITHROL GMS 40 MBAL | 3.9 | Glyceryl stearate |
| SP CRODACOL C90 MBAL | 1.9 | Cetyl Alcohol |
| SP BRIJ CS12 MBAL | 1.2 | Ceteareth-12 |
| SP BRIJ CS20 MBAL | 1.2 | Ceteareth-20 |
| Cetiol V | 3.9 | Decyl Oleate |

Phase B

| | | |
|---|---|---|
| Aqua | 63.6 | Water |
| Glycerine Vegetale PH | 4.9 | Glycerine |
| Kathon CG | 0.1 | Methylchloroisothiazolinone |

Phase C

| | | |
|---|---|---|
| REACH 103 | 19.4 | Aluminum chlorohydrate |

Phase D

| | |
|---|---|
| Extract according to | 3.0 |
| the invention | |

### Protocol

Weigh out phase A and heat. Weigh out phase B, homogenise and heat. Mix phase B into phase A under stirring. Weigh out phase C. Add phase C to phase B+A, with stirring. Allow to cool. Weigh phase D and add to cooled phase C+B+A, under stirring.

### Example 2: Deodorant Spray

TABLE 17

| | | |
|---|---|---|
| Raw materials | % | INCI name |

Phase A

| | | |
|---|---|---|
| Ethanol 96% | 60.4 | Alcohol |

Phase B

| | | |
|---|---|---|
| SP Crodamol IPM MBAL | 1.5 | Isopropyl Myristate |

Phase C

| | | |
|---|---|---|
| D-Panthenol | 0.1 | Panthenol |

Phase D

| | |
|---|---|
| Extract according to | 3.0 |
| the invention | |

Phase E

| | |
|---|---|
| Propellent gas | 35 |

### Protocol:

Weigh out phase A and add phase B, under stirring. Add phase C to phase B+A, under stirring. Add phase D to phase C+B+A, under stirring. In a container, add phase E to phase D+C+B+A.

### Example 3: Face and Body Balm

TABLE 18

| | | |
|---|---|---|
| Raw materials | % | INCI name |

Phase A

| | | |
|---|---|---|
| Water | QSP | Aqua |
| Sorbate de potassium | 0.10 | Potassium sorbate |

Phase B

| | | |
|---|---|---|
| Incroquat ™ Behenyl TMS-50 | 4.00 | Behentrimonium Methosulfate |
| | | (and) Cetyl Alcohol (and) |
| | | Butylene Glycol |
| Crodacol ™ CS90 | 3.00 | Cetearyl Alcohol |
| Crodamol ™ CP | 6.00 | Cetyl Palmitate |
| Crodamol ™GTIS | 4.00 | Triisostearin |
| NG Shea Unsaponifiable ™ | 0.50 | Butyrospermum Parkii (Shea) |
| | | Butter (and) Butyrospermum |
| | | Parkii (Shea) Butter |
| | | Unsaponifiables |
| DI Alpha Tocopherol | 0.20 | Tocopherol |

Phase C

| | | |
|---|---|---|
| Glycerin | 5.00 | Glycerin |
| Phenoxyethanol | 0.80 | Phenoxyethanol |

Phase D

| | |
|---|---|
| Extract according to | 2.00 |
| the invention | |

Phase E

| | | |
|---|---|---|
| Water | 1.00 | Aqua |
| Sodium Hydroxide 30% | 0.10 | Sodium Hydroxide |

### Protocol:

Weigh out phase A and heat to 85 °C in a bain-marie. Weigh out phase B and heat to 85 °C in a bain-marie. Add phase B to phase A, stirring vigorously. Weigh phase C and add to phase A+B with stirring. Add phase D to phase A+B+C at around 45 °C and homogenize. Weigh phase E and adjust the pH with it.

### Example 4: Body Cream

TABLE 19

| | | |
|---|---|---|
| Raw materials | % | INCI name |

Phase A

| | | |
|---|---|---|
| Water | qs | Aqua |
| Xanthan gum | 0.40 | Xanthan gum |
| Arlacel LC ™ | 5.00 | Sorbitan stearate (and) |
| | | Sorbityl laurate |
| Glycerin | 1.50 | Glycerin |

Phase B

| | | |
|---|---|---|
| Caprylic/Capric | 10.00 | Caprylic/Capric Tryglyceride |
| Tryglyceride | | |
| Stearyl Alcohol | 1.50 | Stearyl Alcohol |

Phase C

| | | |
|---|---|---|
| Euxyl PE9010 | 0.1 | Phenoxyethanol (and) |
| | | Ethylhexylglycerin |
| Extract according to | 2.0 | |
| the invention | | |

### Process:

Weigh the water and glycerine and add the xanthan gum. Mix until completely dispersed. Add the Arlacel™ LC and heat. Homogenise and leave to stand. Weigh phase B. Add phase B to phase A, with stirring. Weigh phase C, then add phase C to phase A+B when the temperature is below 40 °C. Homogenise.

Examples of Other Ingredients that May be Added to these Formulations:

Crodarom® Nordic Cotton: active ingredient marketed by Crodarom, to help strengthen the skin's barrier function, soothe the most sensitive skin and moisturise and protect the hair.

Phytessence™ Blue Daisy: Ingredient marketed by Crodarom, soothes sensitive skin and reduces feelings of discomfort by acting on the synthesis of inflammation mediators.

Crodarom® Bamboo Tea EC: ingredient marketed by Crodarom, stimulates cellular metabolism while maintaining the skin's moisture level, and protects the skin from external aggression.

1. A method of treating body odour in a mammal, comprising contacting skin of a mammal in need thereof with a Sichuan berry extract of Zanthoxylum bungeanum or Zanthoxylum simulans species.

2. The method according to claim 1, wherein the treatment is furthermore an anti-aging treatment and/or a moisturizing treatment.

3. (canceled)

4. The method according to claim 1, wherein the treatment acts on the lipase activity of bacteria while preserving the bacterial flora.

5.-11. (canceled)

12. The method according to claim 1, wherein the extract is obtained by extraction under reflux, by maceration, decoction, infusion, leaching, subcritical or supercritical fluid, means of ultrasound or microwaves, percolation, digestion, cryoextraction or enzymatic digestion.

13. The method according to 12, wherein the extract is obtained by digestion of Sichuan berry in aqueous, alcoholic, hydroalcoholic, glycolic or lipidic solvent, or a mixture thereof.

14.-15. (canceled)

16. The method according to claim 1, wherein the treatment is a holistic treatment combining olfactory, beautifying, sensorial and emotional effects.

17. (canceled)

18. The method according to claim 16, wherein the beautifying effect is an anti-ageing and/or moisturizing effect, and/or reducing shiny appearance of the skin.

19. The method according to claim 16, wherein the sensorial effect is a soothing effect.

20. The method according to claim 16, wherein the emotional effect is a well-being effect.
